(19) United States
(12) Patent Application Publication
(10) Pub. No.: US 2024/0242354 A1
(43) Pub. Date: Jul. 18, 2024

(54) CONTROL METHOD FOR DETECTION SYSTEM

(71) Applicant: InnoCare Optoelectronics Corporation, Tainan City (TW)

(72) Inventor: Yu-Heing Chen, Tainan City (TW)

(73) Assignee: InnoCare Optoelectronics Corporation, Tainan City (TW)

(21) Appl. No.: 18/537,749

(22) Filed: Dec. 12, 2023

(30) Foreign Application Priority Data
Jan. 18, 2023 (CN) 202310059279.X

(51) Int. Cl. G06T 7/00

(52) U.S. Cl. CPC G06T 7/0014; G06T 2207/10116; G06T 2207/20081; G06T 2207/20084

(57) ABSTRACT

A control method for a detection system. The detection system includes a detection device. The detection device includes multiple scanning lines. The control method includes the following steps: first image data is generated through the detection device, and at least a part in the first image data corresponds to a key area; a first part corresponding to the key area in the scanning lines is controlled by the detection device in a first scanning manner; a second part corresponding to an area other than the key area in the scanning lines is controlled by the detection device in a second scanning manner. Second image data is generated by the detection device. A scanning frequency of the first scanning manner is lower than a scanning frequency of the second scanning manner.

### CROSS-REFERENCE TO RELATED APPLICATION

This application claims the priority benefit of China application serial no. 202310059279.X, filed on Jan. 18, 2023. The entirety of the above-mentioned patent application is hereby incorporated by reference herein and made a part of this specification.

### BACKGROUND

### Technical Field

The disclosure relates to a control method, in particular to a control method for a detection system.

### Description of Related Art

The image data transmission capability of general detection equipment is limited by the data transmission bandwidth between the detection equipment and external computer equipment. When the detection equipment needs to operate in the mode of capturing high resolution images, the detection equipment will output a huge amount of image data. Therefore, reducing the frame rate of the image data outputted from the detection equipment to the external computer equipment is required to reduce the amount of data transmitted, which will result in a decrease in data transmission efficiency. Conversely, when the detection equipment needs to operate in the mode with high data transmission efficiency, the image data transmission outputted from the detection equipment to the external computer equipment has to reduce the image resolution to reduce the amount of outputted image data, which makes the image quality obtained by the external computer equipment poor.

### SUMMARY

The disclosure provides a control method for a detection system, which may effectively take into account image transmission efficiency and/or image quality.

The control method of the disclosure is adapted for a detection system including a detection device. The detection device includes multiple scanning lines. The control method includes the following steps: first image data is generated by the detection device, and at least a part in the first image data corresponds to a key area; a first part corresponding to the key area in the scanning lines is controlled by the detection device in a first scanning manner; a second part corresponding to an area other than the key area in the scanning lines is controlled by the detection device in a second scanning manner; and second image data is outputted by the detection device, and a scanning frequency of the first scanning manner is lower than a scanning frequency of the second scanning manner.

Based on the above, the control method for the detection system of the disclosure may perform image scanning in different scanning methods for the key area and the area other than the key area of the scanned image, so that the part corresponding to the key area in the image data may maintain higher image resolution, and the part corresponding to the area other than the key area in the image data may reduce the image resolution to maintain the image transmission efficiency.

In order to make the above-mentioned features and advantages of the disclosure comprehensible, embodiments accompanied with drawings are described in detail as follows.

### BRIEF DESCRIPTION OF THE DRAWINGS

FIG. 1 is a schematic circuit diagram of a detection system according to an embodiment of the disclosure.

FIG. 2 is a flowchart of a control method for a detection device according to an embodiment of the disclosure.

FIG. 3A is a schematic diagram of a first image displayed according to the first image data in an embodiment of the disclosure.

FIG. 3B is a schematic diagram of another first image displayed according to another first image data in an embodiment of the disclosure.

FIG. 4 is a flowchart of a control method for a detection system according to an embodiment of the disclosure.

FIG. 5 is a schematic diagram of a gate driving circuit and a pixel array according to an embodiment of the disclosure.

FIG. 6A is a schematic circuit diagram of a pixel array according to an embodiment of the disclosure.

FIG. 6B is a signal waveform diagram of multiple scanning signals of a pixel array according to an embodiment of the disclosure.

FIG. 7A is a data schematic diagram of a part corresponding to the area other than the key area in the second image data according to an embodiment of the disclosure.

FIG. 7B is a data schematic diagram of a part corresponding to the key area in the second image data according to an embodiment of the disclosure.

FIG. 7C is a data schematic diagram of a part corresponding to the area other than the key area in the output image data according to an embodiment of the disclosure.

### DESCRIPTION OF THE EMBODIMENTS

Reference is now made in detail to exemplary embodiments of the disclosure, and examples of the exemplary embodiments are illustrated in the accompanying drawings. Wherever possible, the same reference numerals are used in the accompanying drawings and descriptions to refer to the same or similar parts.

Throughout the description of the disclosure and the appended claims, certain terms may be used to refer to specific elements. People skilled in the art should understand that electronic device manufacturers may refer to the same components by different names. The disclosure does not intend to distinguish between components that have the same function but have different names. In the following description and claims, the words “comprising” and “including” are open-ended words, and thus should be interpreted as meaning “including but not limited to . . . . ”

Directional terms referred to herein, such as “up”, “down”, “front”, “rear”, “left”, “right”, etc., merely refer to directions of the accompanying drawings. Therefore, the directional terms are used to illustrate rather than limit the disclosure. In the accompanying drawings, various drawings show the general features of methods, structures and/or materials used in the particular embodiments. However, these drawings should not be interpreted as defining or limiting the scope or nature covered by these embodiments. For example, the relative sizes, thicknesses and positions of various layers, regions and/or structures may be reduced or enlarged for clarity.

In some embodiments of the disclosure, terms related to bonding and connection, such as “connection”, “interconnection”, etc., unless otherwise specified, may mean that two structures are in direct contact, or may also mean that two structures are not in direct contact, and there are other structures disposed between these two structures. And the terms about joining and connecting may also include the situation that both structures are movable, or both structures are fixed. In addition, the term “coupled” includes any direct or indirect electrical connection means. In the case of a direct electrical connection, the endpoints of elements on two circuits are directly connected or connected to each other by a conductor segment. In the case of an indirect electrical connection, there are switches, diodes, capacitors, inductors, resistors, other suitable elements, or a combination of the aforementioned elements between the endpoints of the elements on two circuits. However, the disclosure is not limited thereto.

The terms “about”, “equal to”, “equivalent” or “the same”, “essentially” or “substantially” are generally interpreted as within 20% of a given value or range, or as within 10%, 5%, 3%, 2%, 1%, or 0.5% of the given value or range.

Ordinal numbers such as “first”, “second”, etc. used in the description and claims are used to modify elements. The ordinal numbers do not imply and represent that the or these components have any previous ordinal numbers, nor do they represent the order of a certain element and another element, or the order of a manufacturing method. The use of these ordinal numbers is merely used to clearly distinguish an element with a certain name from another element with the same name. The claims and the description may not use the same terms, whereby a first member in the description may be a second member in a claim.

It should be noted that, in the following embodiments, the features of several different embodiments may be replaced, recombined, and mixed to complete other embodiments without departing from the spirit of the disclosure. As long as the features of the various embodiments do not violate the spirit of the disclosure or conflict with each other, they may be mixed and matched at discretion.

Unless otherwise defined, all terms (including technical and scientific terms) used herein have the same meaning as commonly understood by one of ordinary skill in the art to which the disclosure belongs. It is understood that these terms, such as those defined in commonly used dictionaries, should be interpreted as having meanings consistent with the relevant art and the background or context of the disclosure, and should not be interpreted in an idealized or overly formal manner unless otherwise defined in the embodiments of the disclosure.

In the disclosure, the detection device may be a device for detecting light, images, two-dimensional images, stereoscopic images and grayscale images, but is not limited thereto. In the disclosure, the detection device may include electronic elements, and the electronic elements may include passive elements and active elements, such as capacitors, resistors, inductors, diodes, transistors, and the like. The diodes may include light emitting diodes or photodiodes. The light emitting diodes may include, for example, organic light emitting diodes (OLEDs), sub-millimeter light emitting diodes (mini LEDs), micro light emitting diodes (micro LEDs) or quantum dot light emitting diodes (quantum dot LEDs), but are not limited thereto. In the disclosure, the detection device may be an X-ray device, and may be configured to obtain a measurement image (e.g., an X-ray image). The detection device may be used in medical detection, security detection, image recognition and other purposes or fields, but the disclosure is not limited thereto. The X-ray device includes an image measurement module, a processor and a memory. The image measurement module may include a measurer array, and the measurer array includes multiple measurers configured for measuring X-rays. When the image measurement module performs measurement, a measurement target may be disposed between an X-ray light source and the image measurement module, the X-ray light source may irradiate the measurement target, and the image measurement module generates a measurement signal and provides the measurement signal and a measurement image to the processor.

In another embodiment, the detection device may be, for example, an electronic device including a controller, and may output or automatically output a control signal to an image measurement device (e.g., an X-ray device) according to user control. The controller may receive the measurement image transmitted by the image measurement device, and the controller may be a field programmable gate array (FPGA) or a graphics processing unit (GPU) or other suitable elements. In another embodiment, the detection device may further include a memory, and the controller may be configured to execute the modules stored in the memory. The memory may be a dynamic random access memory (DRAM). Hereinafter, the detection device is used as an electronic device for outputting image data to illustrate the disclosure, but the disclosure is not limited thereto.

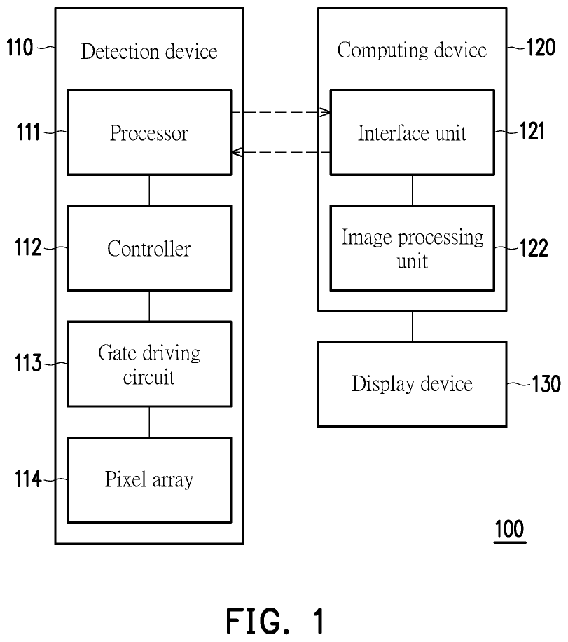

FIG. 1 is a schematic circuit diagram of a detection system according to an embodiment of the disclosure. Referring to FIG. 1, a detection system 100 includes a detection device 110, a computing device 120 and a display device 130. The computing device 120 may be coupled to the detection device 110 and the display device 130 respectively. The detection device 110 may include a processor 111, a controller 112, a gate driving circuit 113 and a pixel array 114. The controller 112 is coupled to the processor 111 and the gate driving circuit 113. The gate driving circuit 113 is coupled to the pixel array 114. The computing device 120 includes an interface unit 121 and an image processing unit 122. The interface unit 121 is coupled to the image processing unit 122. In the embodiment, the detection device 110 may be a flat panel detector (FPD), which may receive an X-ray and convert the X-ray into an electrical signal as detection image data, which may be converted into a detection image. The computing device 120 and the display device 130 may be disposed separately or integrated into an electronic device with data processing, image processing and computing functions such as a personal computer (PC), a laptop, a tablet or a smart phone.

In the embodiment, the gate driving circuit 113 may include multiple gate drivers, and the pixel array 114 includes multiple pixel units and multiple scanning lines. Each of the scanning lines is coupled to plural pixel units, and each of the gate driver is coupled to at least one scanning line. The processor 111 may be, for example, a central processing unit (CPU) to operate the controller 112, so that the controller 112 may control the gate drivers. The gate drivers may respectively drive plural pixel units in each row of the pixel array 114 through the scanning lines. The processor 111 may obtain image data of the detection result of the pixel array 114 and provide the image data to the computing device 120. In the embodiment, the processor 111 may transmit the image data to the interface unit 121 of the computing device 120 through a wired (such as network cable) or wireless (such as Bluetooth or WIFI) transmission method, so that the image processing unit 122 of the computing device 120 may obtain the image data through the interface unit 121, and the image processing unit 122 may perform data processing on the image data. In the embodiment, the interface unit 121 may be realized by a software development kit (SDK), and/or by a physical interface circuit. The image processing unit 122 may for example, include a processor, but is not limited thereto. In addition, it should be noted that the image data referred to in various embodiments of the disclosure may be, for example, X-ray image data, but the disclosure is not limited thereto. The image data referred to in various embodiments of the disclosure may also be image data of other light source types.

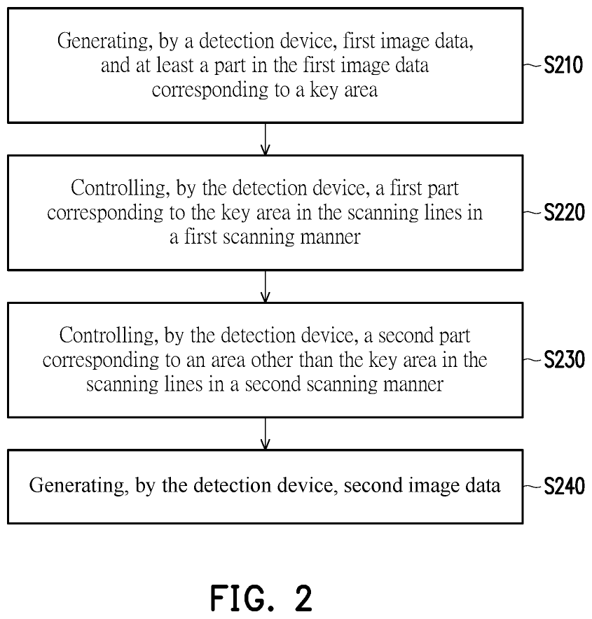

FIG. 2 is a flowchart of a control method for a detection device according to an embodiment of the disclosure. Referring to FIGS. 1 and 2, the detection device 110 may perform operation as the following steps S210 to S240. In the embodiment, the detection device 110 may perform image detection, such as image detection of a human body part by X-rays, to obtain first image data. In step S210, the detection device 110 may generate the first image data, and at least a part in the first image data corresponds to a key area. In the embodiment, the controller 112 may control the gate driving circuit 113 to drive the pixel array 114 to perform image detection, so as to obtain the first image data. The processor 111 may transmit the first image data to the interface unit 121 of the computing device 120. The image processing unit 122 may obtain the first image data through the interface unit 121, and perform data processing on the first image data to determine the location and/or range of the key area. The image processing unit 122 may send back the determination result of the key area to the processor 111, so that the processor 111 may control the gate driving circuit 113 according to the determination result of the key area. In other words, determining the location and/or range of the key area may be based on the data processing result of the first image data, but the disclosure is not limited thereto. In an embodiment, the location and/or range of the key area may also be determined according to an external control signal, and the external control signal may be generated by a user operating an input device (such as a mouse, a keyboard, a touch device installed on a display device or other touch devices, etc.), and received by the processor 111 or controller 112 of the detection device 110 to determine the key area and set the scanning method for the pixel array 114 by the gate driving circuit 113. Alternatively, in another embodiment, the processor 111 of the detection device 110 may also perform data processing on the first image data to determine the location and/or range of the key area. In addition, in the embodiment, the location and/or range of the key area may include a moving object, or other key image objects, so the corresponding key area in the first image data includes moving object image data. In other words, the key area includes the moving object.

In step S220, a first part corresponding to the key area in the scanning lines is controlled by the gate driving circuit 113 of the detection device 110 in a first scanning manner. In step S230, a second part corresponding to the area other than the key area in the scanning lines is controlled by the gate driving circuit 113 of the detection device 110 in a second scanning manner. In step S240, the detection device 110 may generate second image data. In the embodiment, the scanning frequency of the first scanning manner is lower than the scanning frequency of the second scanning manner. The first scanning manner may for example, mean that the gate driving circuit 113 drives the scanning lines (i.e., the first part) corresponding to the key area in a normal one-by-one (scanning-line-by-scanning-line) manner. The second scanning manner may for example, mean that the gate driving circuit 113 simultaneously drives the scanning lines (i.e., the second part) corresponding to the area other than the key area in an image binning manner, so as to reduce the generated image data volume. In this way, the detection device 110 may for example, obtain data of the original image resolution for the part corresponding to the key area in the second image data, and obtain data of lower resolution for the part corresponding to the area other than the key area in the second image data. In other words, the data volume of the second image data obtained by this hybrid scanning method is less, therefore, the data volume of the first image data may be greater than the data volume of the second image data.

Therefore, the detection device 110 may transmit the second image data to the computing device 120 through a way that better image transmission efficiency is maintained. In an embodiment, the image processing unit 122 of the computing device 120 may further adjust the second image data by means of image compensation to improve the image resolution thereof and generate output image data. In this way, the image processing unit 122 may for example, generate output image data with normal image resolution (higher image resolution compared to the second image data), which is then outputted to the display device 130, so that the display device 130 may display a corresponding output image according to the output image data. For this, the data volume of the second image data may be smaller than the data volume of the output image data.

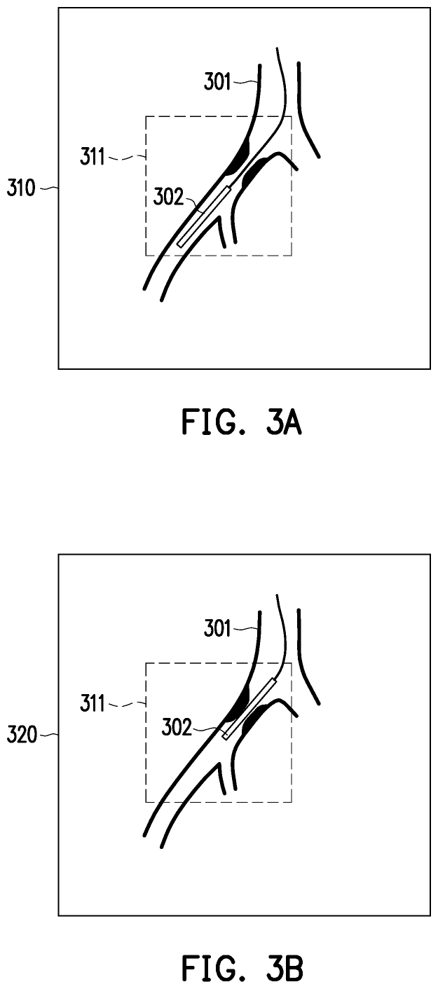

FIG. 3A is a schematic diagram of a first image displayed according to the first image data in an embodiment of the disclosure. Referring to FIGS. 1 to 3A, in the embodiment, an X-ray medical image of a blood vessel is taken as an example, but the disclosure is not limited thereto. The above-mentioned data processing may for example, be performed by the image processing unit 122 or the processor 111 through a trained neural network module (such as a convolutional neural network (CNN)) or a related image recognition module to determine a key area 311 in a first image 310, which means that the image data of the at least a part in the first image data corresponding to the key area is determined, and the key area includes the moving object. As shown in FIG. 3A, the key area 311 may for example, correspond to the area covering a stent 302 within a blood vessel 301 in the first image 310, and the stent 302 may move in the blood vessel 301 and may be identified, set or defined as the moving object. The data processing may identify the location belonging to the moving object (i.e., the stent 302) in the first image 310 to define the location and/or range of the key area 311. In addition, the data processing may include, for example, defining the data content corresponding to the key area in the image data with the minimum rectangular range surrounding the stent 302 (the moving object). Alternatively, in an embodiment, the image processing unit 122 or the processor 111 may directly receive an external control signal generated by the user operating an input device (such as a mouse, a keyboard, a touch device installed on a display device or other touch devices, etc.) to directly set the location and/or range of the key area 311.

FIG. 3B is a schematic diagram of another first image displayed according to another first image data in an embodiment of the disclosure. Referring to FIGS. 1 to 3B, in another embodiment, the above-mentioned data processing may for example, be performed by the image processing unit 122 or the processor 111 to execute an image comparison program, which means comparing the first image data detected at different time points and another first image data. The detection device 110 may for example, perform image detection through the pixel array 114 to obtain image data of another first image 320. The image data of the first image 310 and the another first image 320 may be acquired continuously in time sequence. In this regard, since the location of the stent 302 in the first image 310 and the another first image 320 changes, the image processing unit 122 or the processor 111 may compare the data difference in the image data of the first image 310 and the another first image 320 to determine that the stent 302 is the moving object, and the stent 302 is defined as the data content corresponding to the location and/or range of the key area 311 in the image data.

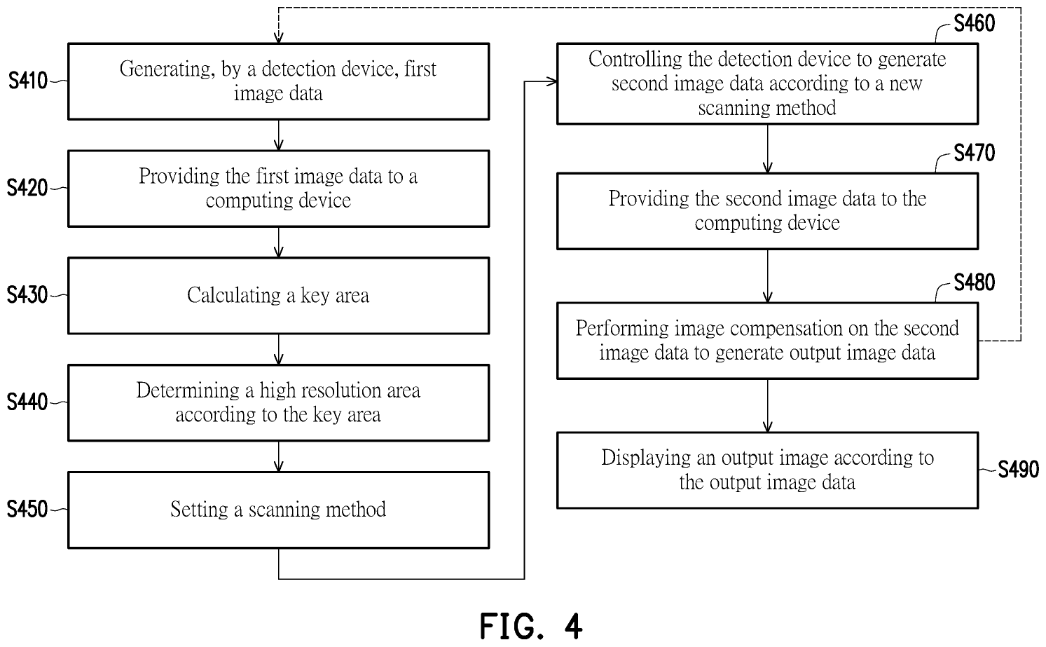

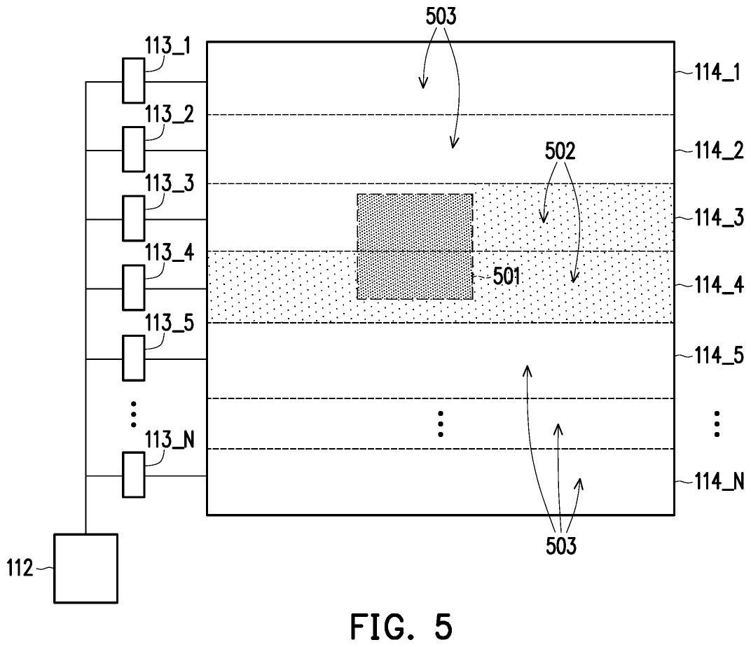

FIG. 4 is a flowchart of a control method for a detection system according to an embodiment of the disclosure. FIG. 5 is a schematic diagram of a gate driving circuit and a pixel array according to an embodiment of the disclosure. Referring first to FIG. 5, the gate driving circuit 113 includes multiple gate drivers 113_1 to 113_N, wherein N is a positive integer. The pixel array 114 may include multiple row pixel groups 114_1 to 114_N, and each row of the row pixel groups 114_1 to 114_N may include at least one scanning line and be coupled to multiple pixel units. The gate drivers 113_1 to 113_N are respectively coupled to the controller 112 and the scanning lines to control the row pixel groups 114_1 to 114_N. It should be noted that each of the gate drivers 113_1 to 113_N may be configured to drive the corresponding coupled scanning line and control the pixel unit coupled to each of the scanning lines, and the following description uses each of the gate drivers 113_1 to 113_N configured to drive a corresponding row pixel group, and each of the row pixel groups being coupled to multiple pixel units as an example.

Referring to FIGS. 1, 4 and 5, the detection system 100 may perform operation as the following steps S410 to S490. In step S410, the detection device 110 may generate first image data. In step S420, the detecting device 110 provides the first image data to the computing device 120. In step S430, the computing device 120 may calculate a key area 501. In step S440, the computing device 120 may determine a high resolution area according to the key area. As shown in FIG. 5, the key area 501 determined according to the first image data may correspond to a certain location and/or range on the pixel array 114, and the processor 111 (and/or the controller 112) may determine the row pixel group 114_3 and the row pixel group 114_4 covered on the pixel array 114 according to the location and/or range of the key area 501, and define the range corresponding to the row pixel group 114_3 and the row pixel group 114_4 as a high resolution area 502 (or a normal resolution area). In the embodiment, the area outside the high resolution area 502 may be a low resolution area 503.

In step S450, the processor 111 may set a scanning method. In the embodiment, the processor 111 may set the row pixel group 114_3 and the row pixel group 114_4 corresponding to the high resolution area 502 to perform scanning according to the normal scanning frequency, and may set other row pixel groups 114_1 to 114_2 and 114_5 to 114_N corresponding to the low resolution area 503 to perform scanning in an image binning manner. In step S460, the detection device 110 is controlled to generate second image data according to a new scanning method. In step S470, the detection device 110 may provide the second image data to the computing device 120. In step S480, the computing device 120 may perform an image compensation program on the second image data to generate output image data. The image compensation program may for example, be an image decompression program. In step S490, the computing device 120 may output the image data to the display device 130, so that the display device 130 may display a corresponding output image according to the output image data. It should be noted that the detection system 100 may re-perform step S410 after performing step S480 according to a preset time length or after displaying a predetermined number of frames of images, so as to re-determine the key area. Therefore, the detection system 100 of the disclosure may automatically update the location of the key area corresponding to the moving object, so as to adaptively adjust the location and/or range of the high resolution area 502 and the low resolution area 503 in the image data.

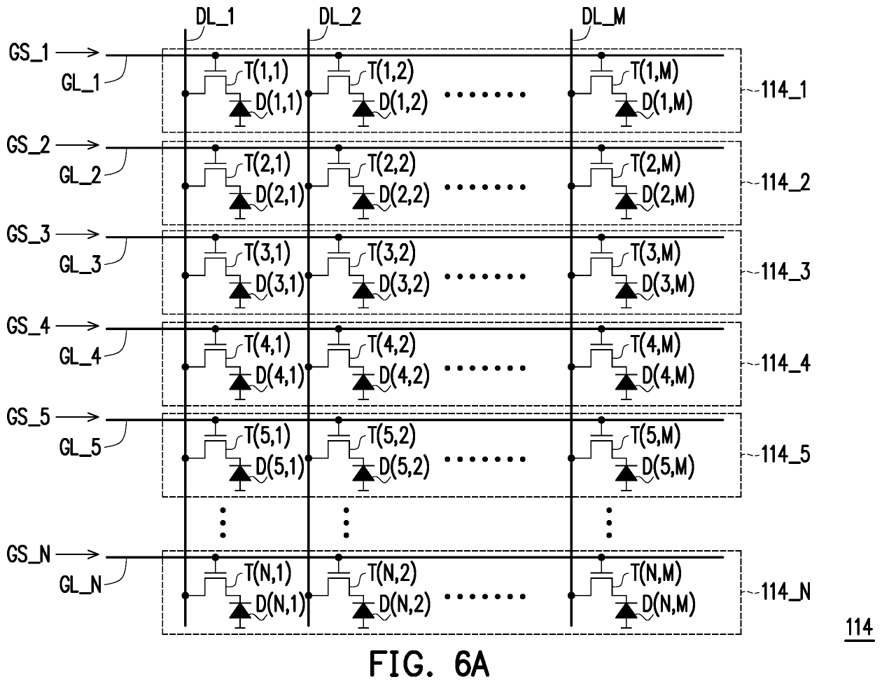

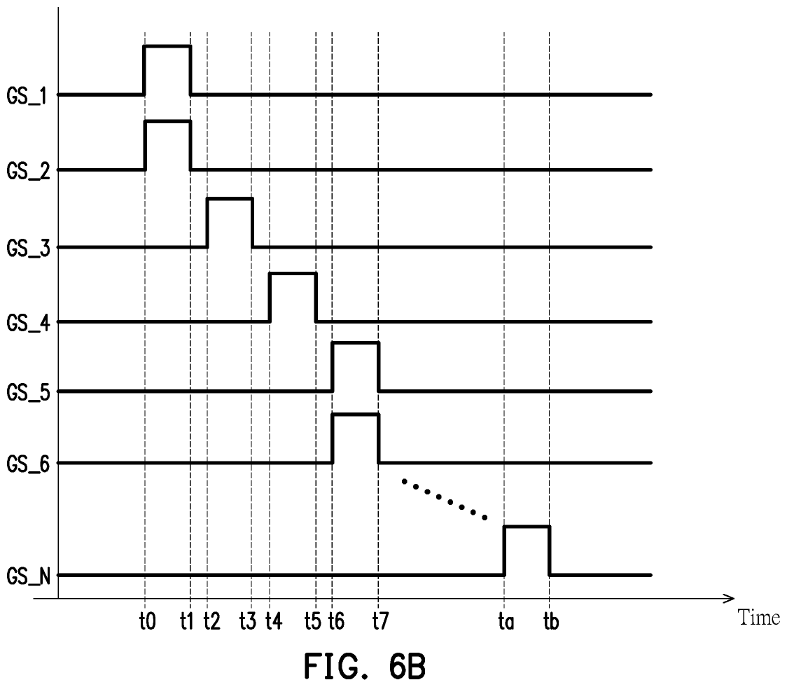

FIG. 6A is a schematic circuit diagram of a pixel array according to an embodiment of the disclosure. FIG. 6B is a signal waveform diagram of multiple scanning signals of a pixel array according to an embodiment of the disclosure. Referring to FIGS. 5, 6A and 6B, the implementation details of scanning in the manner of image binning in the above-mentioned embodiments are described below. In the embodiment, the pixel array 114 includes multiple scanning lines GL_1 to GL_N, multiple data lines DL_1 to DL_M and multiple pixel units, wherein M is a positive integer. Each of the pixel units includes at least one switch element and one electronic element. In an embodiment, the switch element may for example, be a transistor, and the electronic element may for example, be a photodiode, but not limited thereto. In the embodiment, for example, the pixel unit coupled to the data line DL_1 and the scanning line GL_1 includes a transistor T(1,1) and a photodiode D(1,1). Transistors T(1,1) to T(N,M) may for example, be N-type transistors or P-type transistors, but are not limited thereto. A gate terminal of each of the transistors T(1,1) to T(N,M) is coupled to a corresponding one of the scanning lines GL_1 to GL_N. A first terminal of each of the transistors T(1,1) to T(N,M) is coupled to a corresponding one of the photodiodes D(1,1) to D(N,M). The gate drivers 113_1 to 113_N may output gate control signals GS_1 to GS_N to the scanning lines GL_1 to GL_N. A second terminal of each of the transistors T(1,1) to T(N,M) is coupled to a corresponding one of the data lines DL_1 to DL_M. In the embodiment, the row pixel group 114_1 includes the scanning line GL_1, the transistors T(1,1) to T(1,M) coupled to the scanning line GL_1 and the photodiodes D(1,1) to D(1,M), and the row pixel groups 114_2 to 114_N may be deduced by analogy.

Taking a 2×2 image binning method as an example, as shown in FIGS. 6A and 6B, the row pixel group 114_1 and the row pixel group 114_2 correspond to the low resolution area 503 so the detection device 110 may make the gate control signal GS_1 and the gate control signal GS_2 be at a high voltage level during the period from the same time t0 to time t1, so as to simultaneously turn on the transistors T(1,1) to T(2,M) for simultaneously reading (simultaneously detecting) detection data (that is, the higher scanning frequency) of the photodiodes D(1,1) to D(2,M) of every 2×2 pixel units in the row pixel group 114_1 and the row pixel group 114_2, and be at a low voltage level during the period after the time t1, so as to simultaneously turn off the transistors T(1,1) to T(2,M). Similarly, the row pixel group 114_5 and the row pixel group 114_6 correspond to the low resolution area 503 so the detection device 110 may make the gate control signal GS_5 and the gate control signal GS_6 be at a high voltage level during the period from the same time t6 to time t7, so as to simultaneously turn on the transistors T(5,1) to T(6,M) for simultaneously reading (simultaneously detecting) detection data (that is, the higher scanning frequency) of the photodiodes D(5,1) to D(6,M) of every 2×2 pixel units in the row pixel group 114_5 and the row pixel group 114_6, and be at a low voltage level during the period after the time t7, so as to simultaneously turn off the transistors T(5,1) to T(6,M). By analogy, the detection device 110 may make the gate control signal GS_(N-1) and the gate control signal GS_N be at a high voltage level during the period from the same time ta to time tb, so as to simultaneously turn on the transistors T(N-1,1) to T(N,M) for simultaneously reading (simultaneously detecting) detection data (that is, the higher scanning frequency) of the photodiodes D(N-1,1) to D(N,M) of every 2×2 pixel units in the row pixel group 114_(N-1) and the row pixel group 114_N.

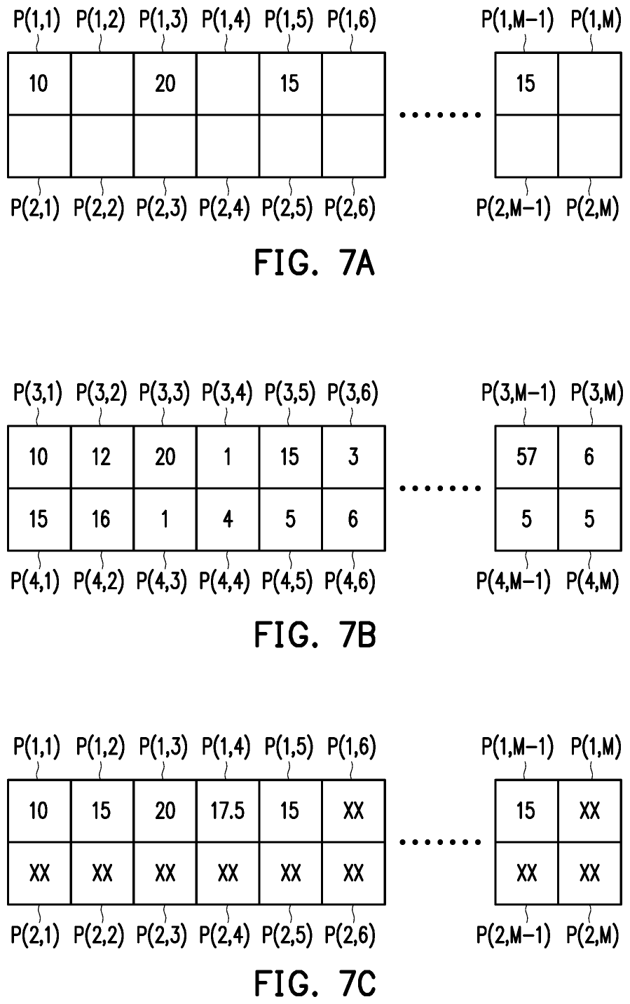

In this way, please refer to FIG. 7A together. FIG. 7A is a data schematic diagram of a part corresponding to the area other than the key area in the second image data according to an embodiment of the disclosure. As shown in FIG. 7A, the data corresponding to pixels P(1,1) to P(2,M) in the second image data generated by the row pixel group 114_1 and the row pixel group 114_2 may be every 2×2 pixel units for a piece of data. For example, the detection results of the pixel unit P(1,1), the pixel unit P(1,2), the pixel unit P(2,1) and the pixel unit P(2,2) may be read at the same time, and for example, an average operation is performed, so as to obtain a pixel value of 10. In an embodiment, the pixel value may be, for example, a grayscale value, but is not limited thereto. In an embodiment, an interpolation operation or a weighting operation may be performed to obtain the pixel value. By analogy, the data volume of the part corresponding to the area other than the key area in the second image data may be, for example, reduced to ¼. For another example, if scanning is performed in a 3×3 image binning manner, the data volume of the part corresponding to the area other than the key area in the second image data may be, for example, reduced to 1/9.

As shown in FIGS. 6A and 6B, the row pixel group 114_3 and the row pixel group 114_4 correspond to the high resolution area 502 so the detection device 110 may make the gate control signal GS_3 and the gate control signal GS_4 be at a high voltage level during the period from time t2 to time t3 and the period from time t4 to time t5 respectively to sequentially turn on the transistors T(3,1) to T(3,M) and the transistors T(4,1) to T(4,M) for sequentially reading (sequentially detecting) detection data (that is, the lower scanning frequency) of the photodiodes D(3,1) to D(4,M) of each pixel unit in the row pixel group 114_3 and the row pixel group 114_4.

As shown in FIGS. 6A and 6B, the time (such as time t1 to time t2, time t3 to time t4, time t5 to time t6 . . . ) between the time when a high voltage level is cut off (such as time t1, time t3, time t5 . . . ) and the time when the next high voltage level is turned on (such as time t2, time t4, time t6 . . . ) is a blanking period.

In this way, please refer to FIG. 7B together. FIG. 7B is a data schematic diagram of a part corresponding to the key area in the second image data according to an embodiment of the disclosure. As shown in FIG. 7B, the data corresponding to the pixel units P(3,1) to P(4,M) in the second image data generated by the row pixel group 114_3 and the row pixel group 114_4 may be each of the pixel units having a piece of data (i.e. the pixel value).

In other words, if the high resolution area 502 occupies ⅓ of the area, and the low resolution area 503 occupies ⅔ of the area, the data volume of the second image data may be reduced by half (1×⅓+¼×⅔=0.5). Therefore, the frame rate at which the detection device 110 transmits the second image data to the computing device 120 may for example, be doubled.

Next, the computing device 120 may perform an image compensation program on the second image data, so as to perform data compensation on the data of the part corresponding to the area other than the key area in the second image data, and generate output image data. Please refer to FIG. 7C together. FIG. 7C is a data schematic diagram of a part corresponding to the area other than the key area in the output image data according to an embodiment of the disclosure. The image processing unit 122 of the computing device 120 may generate the pixel value (“15”) of the pixel unit P(1,2) according to the average of the pixel value (“10”) of the pixel unit P(1,1) and the pixel value (“20”) of the pixel unit P(1,3). The computing device 120 may generate the pixel value (“17.5”) of the pixel unit P(1,4) according to the average of the pixel value (“20”) of the pixel unit P(1,3) and the pixel value (“15”) of the pixel unit P(1,5). By analogy, the computing device 120 may perform data compensation on the data of the part corresponding to the area other than the key area in the second image data, so as to improve image resolution thereof. However, the disclosure does not limit the manner of image compensation and image data compensation. In an embodiment, the image processing unit 122 of the computing device 120 may perform numerical interpolation or other image compensation means. Therefore, the display device 130 of the detection system 100 of the disclosure may display the output image data of normal image resolution (or higher image resolution), and also achieve better image transmission efficiency. More importantly, in the output image data displayed by the display device 130 of the detection system 100 of the disclosure, the image content without image distortion may be maintained for the key area of the image (that is, the area corresponding to the moving object).

To sum up, the control method for the detection device disclosed in the disclosure may first automatically determine the key area in the image data, so that different row pixel groups corresponding to the key area and the non-key area in the subsequent pixel array may be scanned according to different scanning frequencies, the part corresponding to the non-key area in the image data generated by the detected pixel array may have a lower image data volume, and a normal or higher image data volume may be maintained for the part corresponding to the key area. Therefore, the control method for the detection device of the disclosure may enable the detection device to realize the advantages of both data transmission efficiency and image quality.

Finally, it should be noted that the above embodiments are merely used to illustrate the technical solutions of the disclosure, but not to limit the technical solutions of the disclosure. Although the disclosure has been described in detail with reference to the foregoing embodiments, those of ordinary skill in the art should understand that the technical solutions described in the foregoing embodiments may still be modified, or some or all of the technical features thereof may be equivalently replaced. However, these modifications or substitutions do not make the essence of the corresponding technical solutions deviate from the scope of the technical solutions of the embodiments of the disclosure.

What is claimed is:

1. A control method for a detection system, and the detection system comprising a detection device, wherein the detection device comprises a plurality of scanning lines, the control method comprising:
generating, by the detection device, first image data, and at least a part in the first image data corresponding to a key area;
controlling, by the detection device, a first part corresponding to the key area in the plurality of scanning lines in a first scanning manner;
controlling, by the detection device, a second part corresponding to an area other than the key area in the plurality of scanning lines in a second scanning manner; and
generating, by the detection device, second image data,
wherein a scanning frequency of the first scanning manner is lower than a scanning frequency of the second scanning manner.

2. The control method according to claim 1, wherein the key area comprises a moving object.

3. The control method according to claim 2, wherein the key area is determined according to data processing of the first image data, and the data processing comprises determining the key area by a minimum rectangular range surrounding the moving object.

4. The control method according to claim 1, wherein the key area is determined according to an external control signal.

5. The control method according to claim 1, wherein the key area is determined according to data processing of the first image data.

6. The control method according to claim 5, wherein the data processing comprises an image comparison program.

7. The control method according to claim 1, the detection system further comprising a computing device, coupled to the detection device, wherein an image compensation program is performed on the second image data by the computing device to generate output image data.

8. The control method according to claim 7, wherein the image compensation program is to compensate a part corresponding to the area other than the key area in the second image data.

9. The control method according to claim 7, wherein a data volume of the second image data is smaller than a data volume of the output image data.

10. The control method according to claim 1, wherein a data volume of the first image data is larger than a data volume of the second image data.

11. The control method according to claim 1, further comprising:
executing a trained neural network module to determine that the at least a part in the first image data corresponds to the key area.

12. The control method according to claim 1, further comprising:
determining that the at least a part in the first image data corresponds to the key area according to an external control signal.

13. The control method according to claim 1, further comprising:
generating, by the detection device, another first image data; and
comparing data difference between the first image data and the another first image data to determine that the at least a part in the first image data corresponds to the key area.

14. The control method according to claim 1, further comprising:
defining a pixel array to correspond to range of a high resolution area and a low resolution area according to the key area.

15. The control method according to claim 14, wherein the key area corresponds to the high resolution area of the pixel array, and an area other than the high resolution area may be a low resolution area.

16. The control method according to claim 1, wherein the first scanning manner is to drive a plurality of scanning lines corresponding to the key area in a normal line-by-line driving manner.

17. The control method according to claim 1, wherein the second scanning manner is to simultaneously drive a plurality of scanning lines corresponding to an area other than the key area in an image binning manner.

18. The control method according to claim 1, wherein the detection device is a flat panel detector.

19. The control method according to claim 1, wherein the first image data and the second image data are X-ray image data.

20. The control method according to claim 1, wherein a part corresponding to the key area in the first image data comprises moving object image data.
